(19) United States
(12) Patent Application Publication
(10) Pub. No.: US 2008/0275397 A1
(43) Pub. Date: Nov. 6, 2008

(54) Low pierce force needle port

(76) Inventor: Michael J. Bonnette, Minneapolis, MN (US)

Correspondence Address: GREGORY L BRADLEY;MEDRAD INC, ONE MEDRAD DRIVE, INDIANOLA, PA, 15051, US

(21) Appl. No.: 10/930,528

(22) Filed: Aug. 31, 2004

(51) Int. Cl. A61M 5/32

(52) U.S. Cl. 604/117

(57) ABSTRACT

A low pierce force needle port including a compressible resilient seal puncturable by a needle and compressing apparatus composed of a sealing cap and a sealing gland for compressing the seal. Provision of the compressing apparatus enables the seal to be very thin and still effectively seal before, during, and after needle penetration. Annular beveled and rounded shouldered surfaces on the sealing cap and sealing gland, respectively, serve to guide needles entering and passing through the low pierce force needle port; and a handle, a wide planar area, or other feature of the sealing cap provides a degree of protection against inadvertent needle punctures of the practitioner.

### CROSS REFERENCES TO RELATED APPLICATIONS

This application includes subject matter bearing similarity to subject matter disclosed in commonly assigned co-pending application Ser. No. 10/838,464 filed May 4, 2004, entitled “Gas Inflation/Evacuation System and Sealing System Incorporating a Compression Sealing Mechanism for Guidewire Assembly Having Occlusive Device,” pending.

### BACKGROUND OF THE INVENTION

1. Field of the Invention

The present invention is for a medical device, and more specifically, for a low pierce force needle port for receiving and sealing about a needle used for injection of drugs or medicines into tubes utilized for delivery of drugs or medicines into the body or, alternatively, used to withdraw fluid from the body or from an associated fluid system.

2. Description of the Prior Art

Prior art needle ports incorporated a puncturable seal of rubber or other material requiring a certain force to effect a needle puncture. Some seal materials, such as latex rubber for example, or certain other known rubber materials, would offer variable degrees of penetration resistance depending upon the age and/or thickness of the material. A typical prior art rubber seal could harden, thereby offering an increased puncture resistance; or could harden and become brittle, thereby offering resistance to a needle puncture as well as incurring the possibly of rubber particles detaching themselves from the seal to contaminate the interior of the sealed region into which the drug or medicine was being delivered. Suitable resealing upon needle retraction with hardened or brittle material also was a concern. The present invention offers a seal comprised of long lasting silicone offering long lasting structural integrity.

Prior art seals offered resistance to penetration according to the thickness of the seal material and, as such, the practitioner had very little control over the thickness of a seal, as the seals were attached to the surrounding support structure during the manufacturing process. If the force to overcome the resistance of a seal when using different size needles was objectionable, the practitioner was offered few options. The present invention offers a seal which can be removed and subsequently replaced by another seal of other thickness to meet the requirements of the practitioner.

Also in prior art seals, reliance is made on the properties of the materials of the seals to close a puncture site (e.g., in the case of rubber, the rubber tends to flow back to close any puncture), and thicker seals would close more readily. The seal of the present invention uses the force imparted by a compressing apparatus to close a puncture; that is, squishing the seal causes it to expand radially inwardly and outwardly to close a puncture. The compressing apparatus allows the seal to be much thinner and easier to puncture; and suitable sealing no longer depends solely on the material of the seal, as was the case in the prior art.

Safety of the practitioner is another consideration. Prior art needle ports are small in size and, hence, offer a small overall target for the end of the puncturing needle. As such, inadvertent puncture of the practitioner's fingers by poor coordination or a poorly aimed needle can result. The present invention offers a surround area about an initial target area to provide a zone for misdirected or poorly aimed needles.

### SUMMARY OF THE INVENTION

The general purpose of the present invention is to provide an improved needle port.

According to one embodiment of the present invention, there is provided a low pierce force needle port having a sealing gland, a sealing cap connected to one end of the sealing gland, a fluid connector connected to the other end of the sealing gland, and a resilient seal residing in the sealing gland. The fluid connector is preferably a male Luer connector, but may be any other convenient fluid connector as known in the art. The seal, preferably of silicone or other such long lasting resilient and self-sealing material, resides in a cavity in the sealing gland and is sealingly compressed between an annular planar sealing face at the end of a tubular extension of the sealing cap and a planar sealing seat in the sealing gland. Compressing the silicone allows one to make a thin seal that still seals effectively when punctured because the silicone material is forced together from all sides. While the presently preferred seal material is silicone elastomer, other natural or synthetic rubbers or elastomeric polymers can be utilized, such as resilient foams, fibrous mats, or solid layers, or other forms, composites, or combinations. For example, a fibrous elastomeric mat with a solid layer may be utilized. The seal is removable and may be exchanged for a seal of different thickness or qualities according to the requirements and needs of the practitioner. An annular bevel for guidance of a needle to a passageway and to the seal is located at the proximal portion of the sealing cap. A planar area separating the annular bevel from the peripheral edge of the sealing cap provides spacing of a practitioner's fingers from the area of needle insertion at the annular bevel. The low pierce force needle port can be attached to an intravenous or other fluid system, and used to provide access to deliver medicine, diagnostic medium, or other fluid, or to withdraw fluid from the body or fluid system.

One significant aspect and feature of the present invention is a low pierce force needle port having a sealing gland, a sealing cap, a fluid connector, and a seal.

Another significant aspect and feature of the present invention is a low pierce force needle port including a seal of silicone composition to provide a seal which is readily puncturable by a needle.

Yet another significant aspect and feature of the present invention is a low pierce force needle port including a seal which is long-lived and resistant to the rigors of age.

Still another significant aspect and feature of the present invention is a low pierce force needle port having a compressing apparatus which can vary the degree of compressibility of a seal, thereby enabling the seal to be very thin yet still seal effectively, the thinness of the seal also reducing the pierce force required to puncture it and thus making for a safer construction.

A further significant aspect and feature of the present invention is a low pierce force needle port having an annular bevel leading to a passageway and thence to a seal.

A still further significant aspect and feature of the present invention is a low pierce force needle port which provides a region of separation between the practitioner's fingers and an annular bevel, where such bevel is the first point of needle insertion.

Yet a further significant aspect and feature of the present invention is a low pierce force needle port having an annular rounded shoulder internal to a sealing gland for guidance of a needle into a passageway of a connector tube and along or near the longitudinal axis of the low pierce force needle port.

Another significant aspect and feature of the present invention is a low pierce force needle port having elongated and narrowed internal structure of a sealing cap for the purpose of early and accurate alignment of a needle within the sealing cap to facilitate suitable and more accurate needle alignment distal to the sealing cap within a passageway located in a connector tube.

Another significant aspect and feature of the present invention is a low pierce force needle port having a handle extending from a sealing cap.

Having thus described an embodiment of the present invention and set forth significant aspects and features thereof, it is the principal object of the present invention to provide an improved needle port.

### BRIEF DESCRIPTION OF THE DRAWINGS

Other objects of the present invention and many of the attendant advantages of the present invention will be readily appreciated as the same becomes better understood by reference to the following detailed description when considered in connection with the accompanying drawings, in which like reference numerals designate like parts throughout the figures thereof and wherein:

FIG. 1 is an isometric view of a low pierce force needle port in assembled condition;

FIG. 2 is an exploded view of the low pierce force needle port shown in FIG. 1;

FIG. 3 is an exploded cross section view taken along line 3-3 of FIG. 2;

FIG. 4 is a cross section view of the assembled low pierce force needle port taken along line 4-4 of FIG. 1;

FIG. 5 is a cross section view like FIG. 4 but showing the low pierce force needle port in use with a syringe and a needle as well as with a female Luer connector;

FIG. 6, a first alternative embodiment, is a longitudinal cross section view of a low pierce force needle port incorporating the general structure of the low pierce force needle port of FIGS. 1-5 but having elongated and narrowed structure internal to the sealing cap; and,

FIG. 7, a second alternative embodiment, is an isometric view of a low pierce force needle port incorporating the general structure of the low pierce force needle port of FIGS. 1-5 but in addition including a handle extending from the sealing cap.

### DETAILED DESCRIPTION OF THE PREFERRED EMBODIMENTS

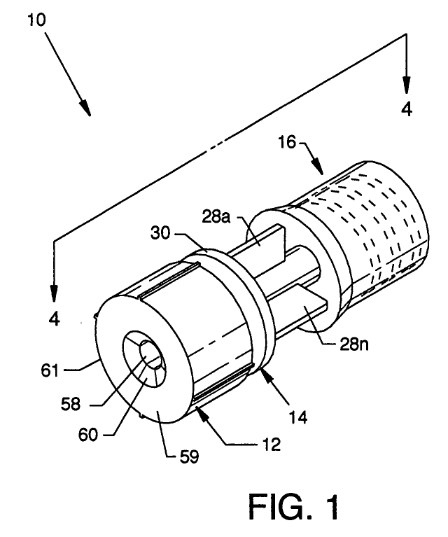

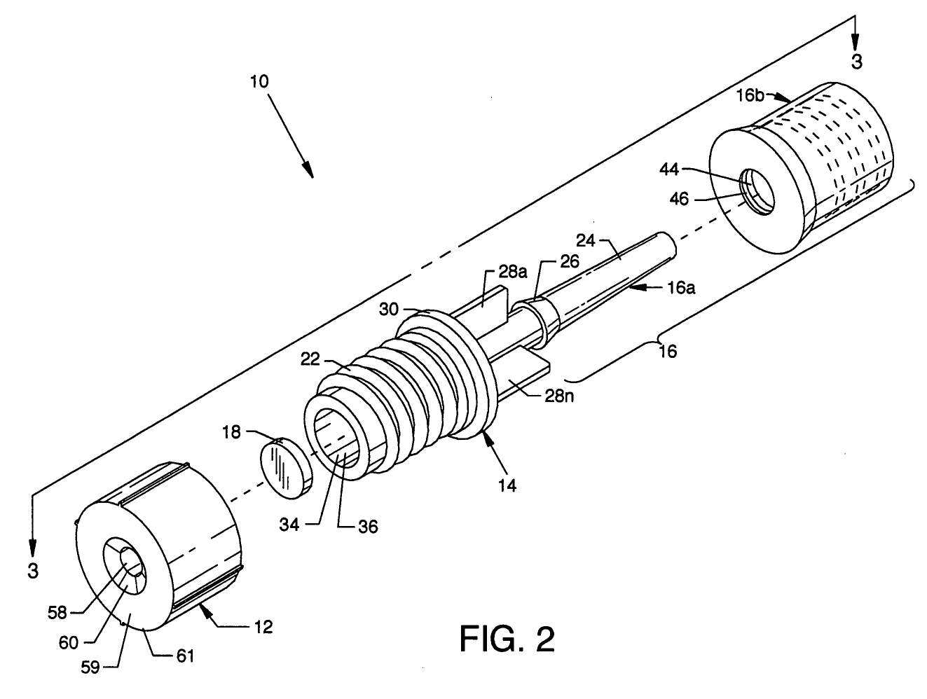

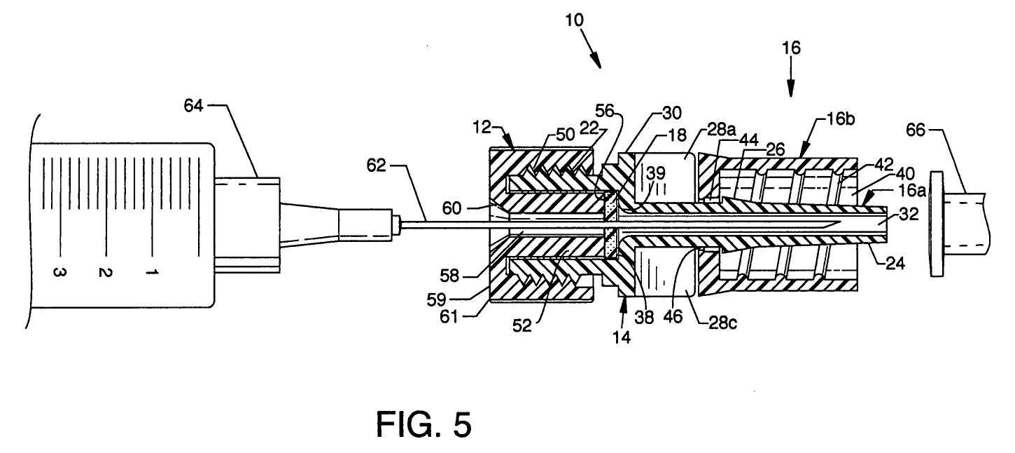

FIG. 1 is an isometric view of a low pierce force needle port 10 in assembled condition, and FIG. 2 is an exploded view of the low pierce force needle port 10 shown in FIG. 1. Immediately visible components in the views include a sealing cap 12, a configured one-piece sealing gland 14, and a fluid connector 16. While in the figures the fluid connector 16 is shown in a preferred configuration as a male Luer connector, any suitable known fluid connector may be utilized. The standard Luer taper connectors are typically used for syringes and infusion sets, but other connectors may be used for particular applications, including custom or proprietary design connectors intended only to connect to other compatible custom or proprietary design connectors in a system. In the figures, fluid connector 16 is further illustrated as a male Luer connector with a threaded locking component; the threaded locking component is preferred to provide a more secure connection, but is not always required. The illustrated male Luer connector is shown as consisting of two components: a Luer taper component 16a which forms a fluid seal with a Luer taper of a mating female Luer connector (item 66, FIG. 5) and a threaded locking component 16b which provides a secure mechanical attachment to tabs or thread portions of a mating female Luer connector (item 66, FIG. 5).

FIG. 2, in addition to showing the sealing cap 12, the sealing gland 14, and the fluid connector 16, shows a seal 18 constructed of a resilient material of suitable composition, preferably silicone, which is self-sealing to an extent with regard to needle punctures and the like and which is substantially unaffected by the rigors of time and temperature. The seal 18 is imperforate, axially compressible, and sealingly radially expandable, and is shaped as a disc having two substantially flat faces substantially parallel to each other and separated by a thickness. The sealing gland 14 serves as a mount for the sealing cap 12 and together with the sealing cap 12 constitutes a compressing apparatus for the seal 18. Sealing gland 14 also serves as a mount for the fluid connector 16. One end of the sealing gland 14 includes external threads 22 for receiving and mounting the sealing cap 12. Located along the longitudinal axis of the sealing gland 14 at the end thereof opposite to the external threads 22 is a connector tube 24 which forms a part of the Luer taper component 16a and which has an annular ramp 26 over which the threaded locking component 16b is mounted by snap engagement and by which the threaded locking component 16b is captured and rotatably retained upon the connector tube 24 proximal to the annular ramp 26 and adjacent to a plurality of support struts 28a-28n extending along a portion of the connector tube 24 and terminating at an annular ridge 30, which is shown to be continuous, but which could be spaced segments, for manual grasping.

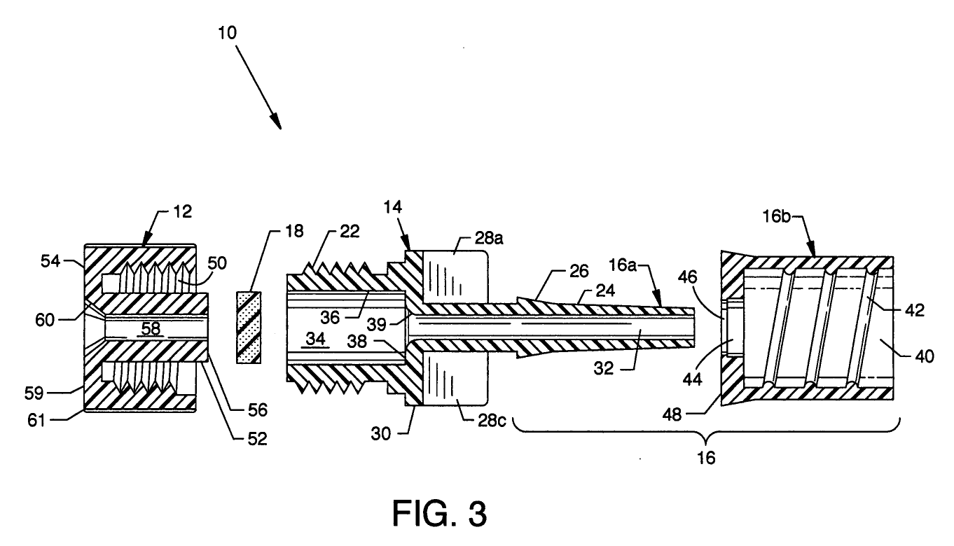

FIG. 3 is an exploded cross section view taken along line 3-3 of FIG. 2. Shown in particular is a proximally located cavity 34 extending along and about the longitudinal axis of the sealing gland 14. The cavity 34 includes a circular peripheral wall 36 intersecting a surface in the form of a sealing seat 38 which is planar in nature and ring-like in shape. A passageway 32 extends partially along and about the longitudinal axis of the sealing gland 14 and within the connector tube 24 in communication with the cavity 34. An annular shoulder 39 bridges the planar sealing seat 38 and the passageway 32 of the connector tube 24. The annular shoulder 39 is shown as a rounded shoulder but could be conical or other tapered shape and functions as a guide to direct the end of a needle into the passageway 32 of the connector tube 24, if required. As stated previously, the fluid connector 16 in this example is a male Luer connector comprising Luer taper component 16a and threaded locking component 16b. The threaded locking component 16b includes an interior cavity 40 which is cylindrical and which includes a raised threaded surface 42 for accommodation and fixation to desired appliances. A hole 44 having an annular ridge 46 is located in the end wall 48 of the threaded locking component 16b. The annular ridge 46 snappingly engages over and about the annular ramp 26 of the connector tube 24 to rotatably retain the threaded locking component 16b upon the connector tube 24 of Luer taper component 16a, as previously described. The sealing cap 12 includes internal threads 50 suitable for threadingly engaging the external threads 22 of the sealing gland 14. A tubular extension 52 extends distally from the end wall 54 of the sealing cap 12 and terminates in an annular planar sealing face 56. The planar sealing seat 38 and the annular planar sealing face 56 are substantially parallel to one another, so that, when the annular planar sealing face 56 of the sealing cap 12 is in contact with one flat face of the resilient seal 18, the annular planar sealing face 56 transmits compressive force to the resilient seal 18 to force the other flat face of the resilient seal 18 against the planar sealing seat 38, thereby compressing the resilient seal. A passageway 58 having a proximally located annular bevel 60 extends from the end wall 54 through the tubular extension 52 and intersects the annular planar sealing face 56. A planar area 59 between the annular bevel 60 and the peripheral edge 61 of the sealing cap 12 offers a wide zone of protection surrounding and extending beyond the annular bevel 60 to distance the practitioner's fingers from a target area which is considered to be the annular bevel 60 co-located with the proximal portion of the passageway 58. The annular bevel 60 functions as a guide to direct the end of a needle into the passageway 58 of the tubular extension 52 for subsequent needle penetration of the seal 18.

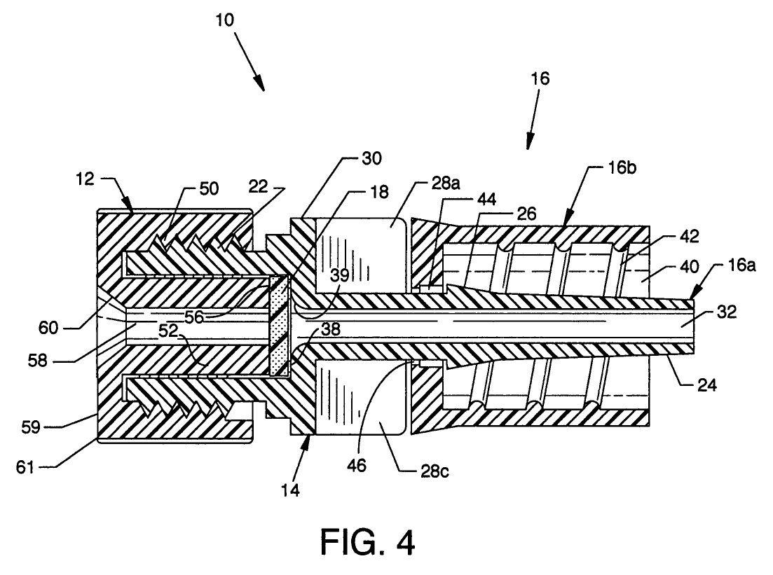

FIG. 4 is a cross section view of the assembled low pierce force needle port 10 taken along line 4-4 of FIG. 1. Shown in particular is the compression of the seal 18 as influenced by the position of the sealing cap 12.

### MODE OF OPERATION

FIG. 5 is a cross section view like FIG. 4 but showing the low pierce force needle port 10 in use with a syringe 64 and a needle 62 as well as with a mating fluid connector illustrated as a female Luer connector 66. The fluid connector 16 at the distal end of the low pierce force needle port 10 can be connected to the mating female Luer connector 66 for delivery of the contents of the syringe 64 through the mating female Luer connector 66 to regions downstream thereof. The mating female Luer connector 66 can be connected at its distal end to communicate with various types of devices, such as an IV port, an IV bag, or other such devices for the delivery or injection of medicines or drugs. FIG. 5 incorporates the elements of FIG. 4 showing the compression of the seal 18. In use, the seal 18 is aligned in the cavity 34 (FIG. 3), preferably in initial contact with the planar sealing seat 38 at one end of the cavity 34, followed by threaded engagement of the sealing cap 12 to the sealing gland 14 to capture the seal 18. Alternatively, the seal 18 could be factory installed and compressed, if desired. The internal threads 50 of the sealing cap 12 threadingly engage the external threads 22 of the sealing gland 14, and the sealing cap 12 is rotatingly advanced with respect to the sealing gland 14 to bring the annular planar sealing face 56 of the tubular extension 52 into intimate contact with the seal 18. Such advancing rotation causes the annular planar sealing face 56 of the sealing cap 12 to forcibly engage the seal 18 to compress the seal 18 between the annular planar sealing face 56 of the sealing cap 12 and the planar sealing seat 38 of the sealing gland 14 to expand the periphery of the seal 18 radially outwardly and inwardly, thereby providing a seal against the circular peripheral wall 36 (FIG. 3) of the sealing gland 14 and against an inserted needle, as well as providing a seal against the planar sealing seat 38 and the annular planar sealing face 56. A needle 62 extending from a syringe 64 or other like device is introduced into the annular bevel 60 and guided by the annular bevel 60, as required, into the passageway 58 of the sealing cap 12, and advanced distally to penetrate and sealingly pass through the seal 18 and into and through the passageway 32 of the connector tube 24 for communication with the mating female Luer connector 66. Preferably, the needle 62 or other like penetrating fluid delivery device substantially maintains a coaxial relationship to the annular bevel 60, the passageway 58, the seal 18, the cavity 34, the passageway 32, the hole 44, the annular ridge 46, and the interior cavity 40 of the threaded locking component 16b of the fluid connector 16, all of which preferably have a direct mutual coaxial relationship or mutual coaxial relationship along a central longitudinal axis. Should a coaxial relationship as described not occur, allowances in the included geometry provide for usability even though a non-coaxial relationship results. Such geometry includes the annular shoulder 39, which alternately could be a bevel, which is available to guide and direct an off-center needle 62 into the passageway 32. Such geometry also includes the annular bevel 60 which is available to guide and direct a needle 62 into the passageway 58 of the sealing cap 12. During active penetration of the seal 18 by the needle 62 and during static penetration of the seal 18 by the needle 62, the resiliency of the seal 18 automatically forces the parted region of the seal 18 to be firmly and sealingly engaged against the sidewall of the needle 62 to maintain a seal against the needle 62 and to maintain the sealed integrity of the passageway 32 into which the needle 62 has been urged. Subsequent to delivery of the medicine or drug from the syringe 64 and needle 62, the retraction of the needle 62 from the passageway 32 and from the seal 18 is followed by self-closure of the puncture of the seal 18 as caused by the resiliency of the seal 18, thereby maintaining the integrity of seal 18 and the integrity of the passageway 32.

Overall, the increased physical size of the low pierce force needle port 10 contributes to desirable attributes of the present invention. Prior art needle ports generally are of a smaller size than that of the present invention. The increased size as provided by the present invention presents the practitioner with an object more readily graspable, especially when wearing surgical gloves, than that object of smaller dimension. Having attributes which promote easier handling is beneficial to providing for a safe and more expedient and accurate puncture involving the low pierce force needle port 10, especially in providing a reduced possibility and occurrence of inadvertent needle stabs inflicted upon the practitioner. Also included in the invention is initial needle guidance to the puncture zone, i.e., the seal 18, such as is provided by the annular bevel 60 of the sealing cap 12. The use of a seal 18 of silicone instead of a conventional rubber seal provides for a more predictable and consistent force for puncturing of the seal 18 by a needle 62, thereby facilitating ease of use. Further, the use of a seal 18 of silicone is preferable to the use of conventional rubber seals because conventional rubber seals tend to deteriorate over a short time span.

A further improvement over prior art needle ports can be provided by the disclosed structure as next set forth. The amount of axial compression and corresponding radial expansion force on seal 18 can be adjusted by the practitioner, if desired. By rotating sealing cap 12 relative to sealing gland 14, the amount of axial compression of seal 18 can be adjusted, resulting in corresponding modification of radial expansion of seal 18 and thereby altering the sealing efficiency of seal 18 against circular peripheral wall 36, sealing seat 38, and sealing face 56. Also, rotation of sealing cap 12 relative to sealing gland 14 will change the overall compressive state of seal 18 which can affect the force required to puncture seal 18 with needle 62 and can affect the frictional force on movement of needle 62 with respect to the punctured seal 18 thereabout. Thus, the practitioner, by rotation of sealing cap 12 relative to sealing gland 14, is provided with means to adjust the sealing and the puncture force, and can adjust the low pierce force needle port to provide increased sealing ability, if required, to reduce leakage, or to adjust the needle penetration force. Further, such adjustments may be made at various points in the procedure; for example, a lower needle penetration force may be desired initially, and an improved seal may be desired later, and a further low needle penetration force may again be desired for subsequent penetrations, and still higher sealing force may be desired after needle withdrawal. These and other similar combinations of adjustments may be made utilizing the features of the present inventive low pierce force needle port, and are not provided by typical prior art devices.

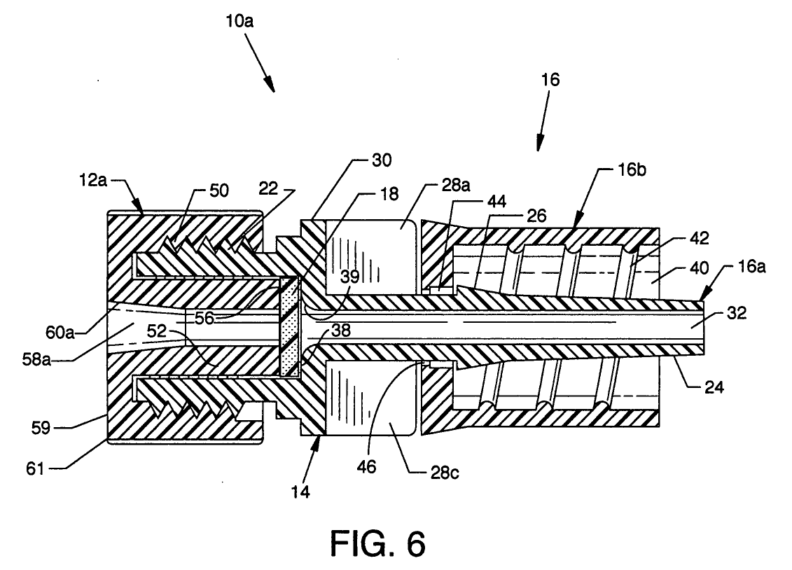

FIG. 6, a first alternative embodiment, is a longitudinal cross section view of a low pierce force needle port 10a incorporating the general structure of the low pierce force needle port 10 of FIGS. 1-5 and subscribing to the teachings thereof but featuring a sealing cap 12a which is similar in many respects to the sealing cap 12 but which has elongated structure for accommodation of a needle. Specifically, the annular bevel 60 of previous figures has been elongated to provide an annular bevel 60a of greater length and having a steeper side truncated conical surface. Further, the previous passageway 58 is replaced by passageway 58a which is correspondingly elongated. Also, the proximally located radius of the annular bevel is reduced to narrow the passageway 58a. Such elongations and narrowing reduces the cross section of the entry zone for a needle to provide early and more accurately guided alignment of a needle within the sealing cap 12a, thereby facilitating suitable and more accurate alignment distal to the sealing cap 12a with the passageway 32 located in the connector tube 24.

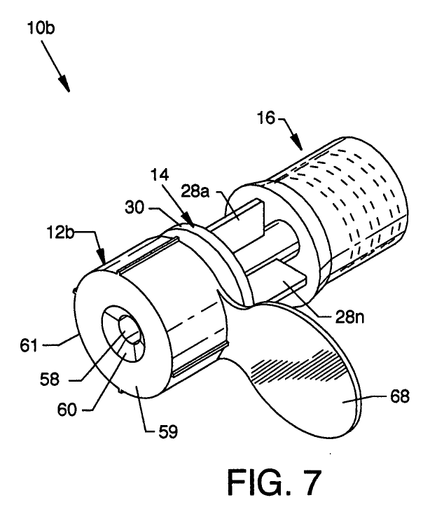

FIG. 7, a second alternative embodiment, is an isometric view of a low pierce force needle port 10b incorporating the general structure of the low pierce force needle port 10 of FIGS. 1-5 and subscribing to the teachings thereof but additionally including a handle 68 extending preferably radially from the periphery of a sealing cap 12b. The health care giver can grasp the handle 68 with a thumb and one or more fingers of one hand, instead of grasping the sealing cap directly with the same appendages of that hand, and can maneuver a needle into and through the sealing cap 12b with the other hand. The distancing of the grasping hand from the general structure of the sealing cap 12b or the other substantial regions of the low pierce force needle port 10b greatly reduces the possibility of accidental needle stabs of the grasping hand.

The invention also includes methods of sealingly accessing a fluid system. One method of accessing a fluid system includes the steps of providing a needle port with a seal and with means for adjusting the compression of the seal, inserting a needle into the needle port and penetrating the seal, and adjusting the compression of the seal.

Various modifications can be made to the present invention without departing from the apparent scope thereof.

It is claimed:

1. A low pierce force needle port comprising:
a. an imperforate resilient seal for receiving and sealing around a needle when inserted therethrough; and,
b. a compressing apparatus for compressing the resilient seal.

2. The low pierce force needle port of claim 1, wherein the resilient seal, when compressed, automatically seals around a needle that penetrates the resilient seal.

3. The low pierce force needle port of claim 2, wherein the resilient seal is formed of silicone.

4. The low pierce force needle port of claim 3, wherein the resilient seal is in the form of a disc having two substantially flat faces substantially parallel to each other and separated by a thickness.

5. The low pierce force needle port of claim 1, wherein the compressing apparatus comprises a sealing cap.

6. The low pierce force needle port of claim 5, wherein the compressing apparatus further comprises a sealing gland having threads that engage threads on the sealing cap.

7. The low pierce force needle port of claim 6, wherein the sealing gland includes a planar sealing seat and the sealing cap includes a planar sealing face that is substantially parallel to the planar sealing seat of the sealing gland so that, when the planar sealing face of the sealing cap is in contact with the resilient seal, the planar sealing face of the sealing cap transmits compressive force to the resilient seal to force the resilient seal against the planar sealing seat of the sealing gland, thereby compressing the resilient seal.

8. The low pierce force needle port of claim 7, wherein the sealing cap further includes a tubular extension and the planar sealing face is on the tubular extension.

9. The low pierce force needle port of claim 8, wherein the sealing cap also includes an end wall having a planar area, and wherein an annular bevel extends from the planar area of the end wall to the interior of the tubular extension.

10. The low pierce force needle port of claim 9, wherein the planar area of the end wall of the sealing cap extends from the annular bevel to an outer peripheral edge of the sealing cap and is sized to provide spacing of the peripheral edge from the annular bevel by a distance which reduces the possibility of needle stabs being inflicted on a user.

11. The low pierce force needle port of claim 6, wherein the sealing gland includes a cavity for receiving the resilient seal, and wherein the cavity includes a sealing seat for the resilient seal.

12. The low pierce force needle port of claim 11, wherein the sealing gland further includes a connector tube having a passageway communicating with the cavity.

13. The low pierce force needle port of claim 12, wherein the sealing gland further includes an annular shoulder at the juncture of the cavity and the passageway of the connector tube.

14. The low pierce force needle port of claim 12, wherein the connector tube includes an annular ramp on its external surface for accommodating and affixing a threaded locking component of a fluid connector.

15. The low pierce force needle port of claim 6, wherein the sealing gland has an annular ridge on its outer surface for manual grasping.

16. The low pierce force needle port of claim 5, wherein the sealing cap includes an end wall having a planar area and wherein a narrow passageway having an entrance surrounded by the planar area extends from the end wall through the seal cap, the narrow passageway having a steep annular bevel at its entrance.

17. The low pierce force needle port of claim 5, wherein the sealing cap includes a handle for manual grasping.

18. The low pierce force needle port of claim 17, wherein the handle extends substantially radially from the periphery of the sealing cap.

19. A sealing assembly comprising:
a. a sealing gland having an externally threaded end and a cavity opening into the sealing gland from the externally threaded end and terminating in an annular, substantially planar sealing seat;
b. a sealing cap having internal threads for cooperating with the external threads of the sealing gland and having a tubular extension for entering into the cavity of the sealing gland, the tubular extension terminating in an annular, substantially planar sealing face; and,
c. a seal in the form of an imperforate disc located between the sealing seat of the sealing gland and the sealing face of the sealing cap, the disc being compressible by the sealing seat and sealing face engaging opposite sides thereof.

20. The sealing assembly of claim 19, wherein the seal is made of silicone.

21. The sealing assembly of claim 19, wherein the sealing cap further includes an end wall and a central annular bevel leading from the end wall into the tubular extension.

22. The sealing assembly of claim 19, wherein the sealing cap includes a handle for manual grasping.

23. The sealing assembly of claim 22, wherein the handle extends substantially radially from the periphery of the sealing cap.

24. A method of accessing a fluid system, comprising the steps of:
a. providing a low pierce force needle port having an imperforate resilient seal for receiving and sealing around a needle when inserted therethrough, and having adjustable compressing apparatus for compressing the resilient seal; and,
b. adjusting the compressing apparatus to adjust the compression of the resilient seal.

25. A method of accessing a fluid system, comprising the steps of:
a. providing a sealing assembly having a sealing gland and a mating sealing cap each with a central passage and adjustable spacing therebetween, and a resilient imperforate puncturable seal compressible between the sealing gland and the sealing cap and blocking the central passage;
b. puncturing the resilient imperforate puncturable seal by inserting a needle therethrough; and,
c. adjusting the spacing between the sealing gland and the sealing cap, thereby adjusting the compression of the resilient imperforate puncturable seal.
